(19) United States
(12) Patent Application Publication
(10) Pub. No.: US 2012/0116295 A1
(43) Pub. Date: May 10, 2012

(54) Topical Growth Factor Application Utilising a Microneedle Array

(75) Inventors: Elliot Isaacs, London (GB); Toby Cobbledick, London (GB)

(73) Assignee: Pangaea Laboratories Ltd, London (GB)

(21) Appl. No.: 13/293,834

(22) Filed: Nov. 10, 2011

(30) Foreign Application Priority Data
Nov. 10, 2010 (GB) 1018999.1
Jul. 26, 2011 (GB) 1112837.8

(51) Int. Cl. A61M 35/00

(52) U.S. Cl. 604/46

(57) ABSTRACT

The present invention relates to improved topical administration of one or more growth factors for non-surgical, cosmetic use. In particular, a combination of one or more growth factors with a microneedle roller. Specifically, the invention resides in a kit for cosmetic use comprising: one or more growth factors, growth factor mimetics, and/or growth factor receptor agonists formulated for topical application; and means to perform wounding of skin, such as a microneedle array, wherein the wounding is targeted to at least the epidermis and/or dermis of the skin to release endogenous thrombin into dermal and/or epidermal tissue.

The present invention relates to improved topical administration of one or more growth factors for non-surgical, cosmetic use.

Growth factors, scientifically known as cytokines, are becoming increasingly popular for use in cosmetic applications. They are primarily large peptides or proteins that act as natural intercellular signals, controlling most cellular functions including cell proliferation, differentiation, migration and apoptosis. Their varied function may be used to stimulate specific pathways and achieve a wide range of cosmetic aims. Such aims include, but are not limited to: reducing wrinkles, hyper-pigmentation and scars; inhibiting or increasing hair growth; accelerating wound healing; creating and maintaining healthy blood circulation. With such an array of functions it is not surprising that there is a demand for growth factors to be widely available for cosmetic use. However, growth factor use is not merely a matter of administering a growth factor or a mixture of growth factors.

Growth factors are similar to hormones and, in fact, biochemists disagree whether certain molecules are cytokines or hormones. There are a number of classes of cytokines and new cytokines and sub-types of cytokine are still being discovered. Additionally, there are various known cytokine mimetics and cytokine receptor agonists. Therefore, there are many options when considering which growth factor to use and in what combination. However, the effects of known growth factors and their mimics are well defined in the literature. Since most growth factors do not have an effect relevant to any of the aims listed above, choosing a suitable growth factor for a particular cosmetic use is not believed to be particularly onerous. For example, growth factors that potentiate immune responses or have roles in foetal development are clearly not useful in a cosmetic context.

While the choice of growth factor for cosmetic use is relatively straight forward, there is an added complication when administering these molecules as many have more than one function, with some functioning differently in different tissues or organs. For example, a growth factor may increase blood circulation but also increase skin pigmentation. This would be beneficial to maintain healthy blood flow for hair growth, but could cause epidermal hyper-pigmentation if used on the skin. Therefore, care must be taken when considering administration as the growth factor may not necessarily have the intended effect. In fact, many may have detrimental or even harmful effects if not used correctly.

Furthermore, complications are added by the fact that many growth factors interact with each other, for example by acting on the same pathways or regulating the expression of receptors for other growth factors. This means that using a combination of growth factors may result in a better effect, but the wrong combination of growth factors could have no effect at all. So, not only is selecting correct individual growth factors important, but also selecting correct combinations of growth factors.

Even an effective combination of growth factors may not achieve the desired result. Growth factors need to reach their specific receptors in order to have an effect on a cell. If a cell does not express the correct receptor then the growth factor will have no effect. Therefore, growth factors much reach the target tissue in an effective concentration and that tissue must express the correct receptors.

The application of growth factors is also not without difficulties. Growth factors are complex proteins which are susceptible to being broken down by digestive enzymes or denatured by the low pH in the stomach. Furthermore, oral administration requires the use of significant concentrations of active ingredient with an associated increase in likely unwanted side effects. Therefore, for superficial, cosmetic use, oral administration is not a preferred route of administration.

Growth factors are large molecules and so intravenous injection has been suggested by several people skilled in the art. However, intravenous injection is limiting because, as mentioned previously, most growth factors have a variety of effects which may be detrimental to the treatment goal, or even be harmful to the person to whom it is applied. Evidence suggests that repeated intravenous injection of certain growth factors has the potential to create unintended immune responses and worsen a variety of medical disorders. Thus, any intravenous application should be considered a last resort in light of the potential dangers.

Topical administration is the most appropriate route of administration because the effects may be targeted to a particular area of the body. Also, cosmetic use of growth factors is usually highly localised and rarely systemic. Unfortunately, this method of administration is particularly inefficient for large molecules such as growth factors because the skin presents an effective barrier for such large molecules. Several well-documented methods of improving topical application exist, including transdermal delivery patches, iontophoresis, electrophoresis, light and laser therapies, skin-thinning treatments such as corticosteroids and liposomal or nanosomal formulations.

Alternatively, mesotherapy, microneedle arrays and shallow hypodermic injections have been used to increase penetration of topical formulations that include large molecules. Such methods, known as controlled or targeted wounding, create a micro-pore or channel in the skin, thereby providing an increased surface area and enabling increased absorption. An example of a suitable device is described in GB 2472778, in which a roller with microneedles projecting radially from a rolling barrel is attached to a handle. The device is rolled along a surface of hair or bare skin with the microneedles making punctures in the skin surface.

Mesotherapeutic devices apply multiple injections of cosmetic formulations primarily into the subcutaneous fat. Suitable devices vary widely, from the simplest syringes to specially designed mesotherapy “guns” which are often trigger operated and can be calibrated. It is also possible to use light, especially laser light, to cause injury and this is also considered as a controlled wounding method. However, these methods serve only to increase the amount of active ingredient reaching the target tissue by reducing the barrier presented by the skin. They do not guarantee that the active ingredient will reach the appropriate cellular target or receptors, resulting in the desired cosmetic effect.

The Applicant has found that consideration of the pharmacological target within the skin is particularly important when applying growth factors. While it is acceptable simply to apply a suitable growth factor formulation by topical means, the concentration of growth factor within the formulation will need to be significantly high to ensure that any cosmetic result is seen at all. Thus, products are expensive, due to the high quantities of active ingredient that are required, and inefficient because of the route of administration.

In light of this, the Applicant has devised an improved method for the topical administration of a growth factor or combination of growth factors that ensures the growth factor, or combination of growth factors, not only reaches its target but has a desired effect without undue side effects. Such method enables the formulation of a topical growth factor cosmetic that requires less active ingredient and is more efficient in its intended effect than currently known formulations.

In its broadest scope, the present invention resides in the use of targeted wounding of the skin to sensitise dermal and epidermal tissue to one or more topically applied growth factor, growth factor mimetic, and/or growth factor receptor agonist.

Expressed in another way, the invention resides in the targeted delivery of a topical formulation comprising one or more topical growth factor, the target being at least the dermal and/or epidermal layers of the skin. The invention relates to a specific use of controlled or targeted wounding, particularly using microneedle arrays but also other methods, to improve the results of topical growth factor application beyond what is known and expected from increasing penetration.

Specifically, the present invention is a kit for non-surgical, cosmetic use comprising one or more growth factors, growth factor mimetics, and/or growth factor receptor agonists formulated for topical application; and means to perform controlled and multiple wounding of an area of skin, wherein the wounding is targeted to at least the epidermis and/or dermis of the skin to release endogenous thrombin into dermal and/or epidermal tissue. The terms “controlled wounding” and “targeted wounding” are used interchangeably throughout the specification and are to be read as having the same meaning.

The Applicant has discovered that release of thrombin in the dermis or epidermis enhances the effect of topically applied growth factors. This is achieved by targeted wounding to the skin. In particular, the Applicant has found that simply randomly increasing or decreasing the depth of controlled wounding is insufficient to achieve the desired results. However, directing the wounding to a depth within the skin that results in thrombin release not only provides enhanced delivery of growth factors but also an enhanced effect. The specific depth depends on the target of the growth factors but is generally the dermis or epidermis for application to bare skin and the dermis or vasculature below for application to hair. This is because the pathways required to increase or decrease hair loss generally reside in the dermis. Similarly, the biochemical pathways involved in reducing scarring, hyper-pigmentation and the appearance of wrinkles are found in the dermis.

Thrombin is released in response to wounding; this is particularly obvious when blood is drawn because thrombin is key to the blood clotting cascade. However thrombin is also released and reaches dermal and epidermal tissues even when no blood is visible. When the skin and/or blood vessels are damaged, but not enough to cause visible bleeding, blood vessels dilate and become more porous, leaking serum proteins such as thrombin. While not wishing to be bound by theory, the Applicant believes that thrombin increases the expression of certain growth factor receptors via a pathway known as the protein kinase-C (PKC) dependent pathway, in vascular, dermal and epidermal tissues.

An increase in receptor expression means that any growth factors released or added topically are more likely to bind to a receptor and therefore have an effect. The overall result is improved as the target tissue effectively becomes more sensitive to growth factor signals. It will be appreciated that while the natural activity of endogenous growth factors could be used, the augmentation of endogenous growth factors with topically applied growth factors is preferred.

Thus, a first embodiment comprises controlled or targeted wounding to release thrombin and a topical preparation comprising one or more growth factors, growth factor mimetics, or growth factor receptor agonists.

Controlled or targeted wounding refers to repeated and consistent wounding to achieve a particular end point. In one embodiment, targeted wounding is applied, for example using a single needle or laser, which is inserted into the skin multiple times over an area of skin to be treated, ideally at the same angle and depth of penetration on each puncture. Every wound is identical so the outcome may be predicted.

To ensure repeatability, it is preferred if an array of wounding means, such as a microneedle array, is used. For example, a microneedle array mounted on a roller may have one hundred and ninety-two needles all of identical length. In this way, a single roll forwards creates one hundred and ninety-two identical wounds.

It will be appreciated that the depth of controlled or targeted wounding should be such that effects are seen in the dermis or epidermis according to the result that is desired. The Applicant has found that wounding at depths of around 0.1 mm, 0.2 mm and 0.3 mm are suitable for epidermal treatment, depths of about 0.5 mm and 1.0 mm are suitable for dermal treatment, while depths of about 1.5 mm, 2.0 mm and 3.0 mm are suitable for targeting delivery to the subcutaneous vasculature. While round numbers are listed above, it will be appreciated that the invention encompasses intermediate depths such as 0.15 mm, 0.24 mm 0.37 mm, 0.83 mm, 1.2 mm, 1.34 mm, 1.8 mm and so on. It is also considered that, whilst the numbers listed above are appropriate for certain areas of the body, the relative depth of each skin layer varies in different areas of the body. For example, the dermis is only around 0.3 mm thick on the eyelid, but nearly 3.0 mm thick on the back. Different individuals may also have differing epidermal and dermal thicknesses. Therefore different wound depths will be suitable depending upon the area of the body, as well as the cosmetic use.

In order for the invention to create significant improvements in results, target cells need to express cytokine receptors consistently. By definition, the cytokines used to maintain or improve hair and/or condition increase cellular proliferation. Increased proliferation leads to increased cellular turnover and, therefore, if treatment is successful, those cells with more cytokine receptors will quickly be replaced by new ones. If the wounding is not performed regularly, these new cells will not express the cytokine receptors. If this occurs, the increase in topical cytokine performance will not last. Therefore it is essential that wounding be performed regularly.

Controlled wounding with devices such as microneedle rollers and mesotherapy guns has been developed specifically for regular use. The wounding is of such an extent that inflammation and thrombin release is consistent, local, and tolerable. Wounds heal quickly and without scarring and so are acceptable to the user (Aust, M. C., et al., (2008) Plastic and Reconstructive Surgery 121(4): p. 1421). Deeper or broader wounds, such as by deep peels or a single needle, cause more damage than a user will accept on a regular basis. It should be noted that applying a single needle repeatedly causes a significant amount more damage than a microneedle roller—even one with the same needle length. This is because human error makes the single needle cut a slightly different path on entering and leaving the skin, leaving an area of damage around it. Multiply this small error by the thousands of needle insertions necessary to mimic a microneedle roller and the increase in damage is highly significant.

Another consideration is that the effect of non-controlled wounds will not be consistent. The invention provides a method by which thrombin will be released in the same concentration evenly over the treated area and, therefore, the cytokine receptor up-regulation will be even. Non-controlled or non-targeted wounding may cause increased thrombin as one point, but less at another. Therefore the treatment result will not be consistent. This is particularly significant because, if a high level of thrombin is released farther upstream in the blood circulatory system, this may reduce thrombin availability downstream.

The final consideration is that deeper and/or broader wounding causes a different physiological response from targeted wounding. More extensive wounds correlate to a significant increase in inflammatory cytokines, many of which may act to create the opposite response to the one desired by the present invention. For example, TGF-beta is a cytokine which inhibits cell proliferation and is expressed strongly around deep or wide wounds that may scar, but not around targeted wounds that do not scar (Ferguson M W., & O'Kane S., (2004) Philosophical Transactions of the Royal Society of London. Series B: Biological Sciences 29;359(1445):839-50). This cytokine would act directly in opposition to EGF which is one example considered useful for topical application. This would prevent the therapeutic benefit of EGF. Another physiological difference is that controlled wounds create local hypoxia, which increases thrombin production. However, broader wounds are exposed to oxygen far more rapidly and so any hypoxia will diminish, decreasing thrombin production.

Therefore targeted wounding is necessary to create the correct physiological conditions for thrombin up-regulation of cytokine receptors. Other wounds will not create the same physiological effect, will create conditions detrimental to the treatment goal, and will not be tolerated by users throughout a course of treatment.

It has been found that results of the present invention are further improved by the inclusion of vibrating the means to perform wounding. Where light or laser light is used for targeted wounding, the vibration only has a significant effect if the emitter is in direct contact with the skin. In a preferred embodiment, the vibration is sonic or ultrasound. Vibration using kinetic movement, for example an offset motor, is considered, but is not an ideal embodiment since the movement of the needles in the skin may cause tearing. Vibration, particularly sonic or ultrasound, may increase cell membrane permeability and cell lysis, releasing yet more thrombin than targeted wounding alone, without vibration. In particular, ultrasonic vibration is believed to create cavitation bubbles in the tissue, which increases thrombin release further still. The increased thrombin levels will have a greater effect in increasing growth factor receptors. Thus, in a preferred embodiment, the means to perform controlled wounding include an ultrasound generator and emitter.

The controlled wounding may also contribute to or cause hypoxia in the wound region which may, in itself, also result in the upregulation of thrombin production from dermal macrophages.

In an alternative embodiment, the means to perform controlled wounding may include a device to dispense the one or more growth factors, growth factor mimetics, and/or growth factor receptor agonists.

Growth factors considered for inclusion in the topical formulation of the invention may be selected from one or more of: EGF, hb-EGF, PDGF-aa, PDGF-bb, FGF4, FGF5, FGF6, FGF8, FGF9, IL-1, IL-2, IL-3, IL-4, IL-5, IL-6, IL-7, IL-8, IL-9, IL-10, IFN-gamma, G-CSF, GM-CSF, IGF-1, IGF-2, KGF, SCF, VEGF, PIGF-I, LIF, TNF-alpha, TNF-beta, M-CSF, BMP-4, HGF, NGF, TGF-alpha. Said growth factors are considered from human, recombinant and/or synthetic sources. Recombinant or synthetic growth factors may be derived from one or more of an animal cell, a plant, yeast and/or bacteria. Mimetics or receptor agonists for any of said growth factors are also considered.

Growths factors of particular interest, either alone or in combination, are: EGF, VEGF, hb-EGF, PDGF-aa, PDGF-bb, FGF1, FGF2, KGF, HGF, IGF-1, SCF, TGF-alpha, GM-CSF and IFN-gamma. A further subset of growth factors that are of value as topical cosmetics are VEGF, PDGF, FGF2, KGF, & IGF-1. EGF, either alone or in combination with other growth factors, is of particular interest in cosmetic skin care formulations, while VEGF is preferred for hair care products.

Certain other synergies are proposed which may further improve the results from controlled wounding prior to topical growth factor application.

Controlled wounding stimulates heparin production and many growth factors have increased affinity for their receptors when bound to heparin, particularly hb-EGF and KGF. Controlled wounding will make compositions that include heparin binding growth factors more effective due to increased receptor affinity. Thus, the inclusion of heparin in the topical formulation is advantageous.

In another embodiment, a preparation comprising thrombin may be applied before or after controlled wounding. Additionally or alternatively, the topical preparation comprising one or more growth factors, growth factor mimetics and/or growth factor receptor agonists may further include thrombin.

Controlled wounding may also stimulate other pathways such as the Wnt pathway for stimulating cell proliferation. Wnt, in turn, is believed to stimulate the PKC pathway and therefore growth factor receptor expression. This is believed to be less significant than thrombin but may be utilised where thrombin is present in low concentrations after controlled wounding, such as the upper epidermis. The Wnt pathway will also support the effects of many of the growth factors considered herein. TGF-alpha, EGF, PDGF, IGF-1, HGF and VEGF are of particular interest because these growth factors act on a pathway shared with Wnt and so any or all of them may act positively to reinforce the effects of the other or others listed in the group.

Light, particularly laser light, is additionally thought to have a positive effect on skin and hair condition and so the possibility of using the growth factor formulation with regular laser therapy is also considered. At certain wavelengths, light can stimulate blood circulation and thus the local circulation of any topically applied composition. Accordingly, light, which may be laser light, is also considered for use with the growth factor formulation to improve penetration and distribution of the formulation.

The cellular migration effect of growth factors partially relies on nitric oxide production. Therefore an advantageous embodiment may also include one or more of: an antioxidant, catalase, catalase mimetic, superoxide dismutase, superoxide dismutase mimetic. Together, these molecules act to remove reactive oxygen and nitrogen species, thereby prolonging the duration of the nitric oxide signal.

Possible antioxidants include one or more of, but are not limited to: green tea extract, vitamin E or a vitamin E derivative, vitamin A or a vitamin A derivative, and vitamin C or a vitamin C derivative.

Possible catalases or catalase mimetics are one or more of, but not limited to: copper PCA, and Catalase.

Possible superoxide dismutases are one or more of, but not limited to, the following: Cu/Zn Superoxide Dismutase, Mn Superoxide Dismutase, Lipochroman-6, EUK-134, copper (II) 3,5-diisopropylsalicylate, copper (II) 3,5-dibromosalicylate, copper (II) 3,5-ditertiarybutylsalycilate, propyl 3,4,5-trihydroxybenzoate.

Similarly, other methods of increasing nitric oxide should have a beneficial effect. Therefore, embodiments including nitric oxide synthases, nitric oxide donators, and nitric oxide mimetics, such as any of eNOS, iNOS, minoxidil, tempo, molsidomine and S-nitrosoglutathione, are also considered.

An alternative or additional embodiment may include into the growth factor formulation a UV protective agent to reduce UV damage and free radical formation. Embodiments including UV protective materials have the additional benefit of preventing further skin blemishes such as hyper-pigmentation, to which alopecia sufferers and those with scar tissue may be more susceptible. Various UV protective agents are considered, including but not limited to one or more of the following: propanediol, quaternium-95, polyacrylate-15, titanium dioxide, zinc oxide, ethylhexyl methoxycinnamate, butyl methoxydibenzoylmethane, octyl methoxycinnamate, menthyl anthranilate, homosalate, benzophenone-3 and benzophenone-4.

The anti-apoptotic action of growth factors occurs largely downstream of extra-cellular apoptotic signals. Whilst this has the benefit of being more specific, targeting the apoptotic pathway prior to the intracellular signals does increase the possible amplitude of the effect. Due to this, an advantageous embodiment may include one or more 5 alpha reductase inhibitors for reducing dihydrotestosterone levels such as ketoconazole, finasteride, dutasteride, turosteride, bexlosteride, izonsteride, FCE 28260, SKF 105,111, saw palmetto extract, green tea extract and hydrolysed lupine extract, and/or another means for reducing the effects of dihydrotestosterone such as caffeine.

Stimulation by growth factors may increase circulation and therefore have a beneficial effect as part of many cosmetic preparations when included with other cosmetically active agents. Cosmetically active agents are defined as compounds (natural or synthetic) that have a cosmetic or therapeutic effect on the skin, hair or nails including, but not limited to, lightening agents, darkening agents, anti-acne agents, shine control agents, anti-microbial agents, anti-inflammatory agents, anti-mycotic agents, anti-parasite agents, external analgesic, sunscreens, photo-protectors, antioxidants, keratolytic agents, detergents or surfactants, moisturisers or humectants, nutrients, vitamins, energy-enhancers, growth factors, anti-perspiration agents, astringents, deodorants, hair-removers, firming agents, anti-callous agents and agents for hair, nail and/or skin conditioning. Of particular interest are curcumin, taurine, plant sterols, pine bark extract, red tea, white tea, horsetail extract, marine cartilage, kieslerde, melatonin and mimetics, copper peptides, other growth factors and growth factor mimetics, spironolactone, β-glucan, vitamins C, A, E, B, F, H, K (and derivatives), bacterial filtrates, glucosamine sulphate, or any combination of these.

Whilst the controlled wounding before application of a growth factor composition will inherently improve penetration, an embodiment wherein one or more parts of the composition are encapsulated in nanosomes or liposomes is also considered. Hypodermic injection, use in a mesotherapy device, and other methods to increase penetration such as transdermal delivery patches, iontophoresis, electrophoresis, light and laser therapies, skin-thinning treatments such as corticosteroids are considered in addition to the controlled wounding prior to application.

The vasodilatory effects of several growth factor pathways stimulated may also be useful as part of other growth factor therapies. Autologous growth factor therapies are becoming increasingly popular for alopecia treatment, skincare, and surgical purposes. Any of the embodiments discussed previously should have beneficial effects when applied as part of an autologous growth factor treatment regime. Non-autologous growth factors are currently not widely legalised and used, but any of the embodiments discussed previously may be beneficial as part of these potential future treatments.

Various methods for utilising the kit of the present invention are also considered. In its simplest form, the method comprises topical application of a formulation comprising one or more growth factors, growth factor mimetics, and/or growth factor receptor agonists, and the application of means to perform wounding of skin, wherein the wounding is targeted to at least the epidermis and/or dermis of the skin to release endogenous thrombin into dermal and/or epidermal tissue.

In one embodiment, targeted wounding takes place immediately prior to application of the topical formulation comprising one or more growth factors, growth factor mimetics, and/or growth factor receptor agonists. Alternatively, targeted wounding takes place immediately after application of the topical formulation. Targeted wounding may also take place up to thirty days prior to application of the formulation.

In an alternative embodiment, the method is part of an autologous growth factor therapy procedure such as platelet-rich plasma therapy. In such therapy a sample of the patient's blood is centrifuged and separated so that only the platelet rich portion remains. This portion is then partially re-injected and partially topically applied over a treatment area.

Of particular interest is use of the method or kit to reduce hair loss, and/or to maintain or improve hair growth. In such a use, application of the growth factor formulation is ideally applied after targeted wounding.

An alternative use of the method or kit is to reduce the appearance of skin blemishes such as hyper-pigmentation and scarring. A further alternative use of the method or kit is to treat or prevent damage to skin cells associated with accumulation of reactive oxygen or nitrogen species such as free radicals. A yet further alternative use of the method or kit is after one or more of cosmetic surgery, hair transplant surgery, implantation of dermal filler and/or surgical implants, and/or autologous growth factor therapy, to reduce recovery time and/or improve the desired cosmetic result. The Applicant has also found that the method and kit have use in the preparation of hair grafts for removal prior to hair transplant surgery.

It will be appreciated that the formulation may be applied prior or immediately prior to targeted wounding, but in a preferred embodiment, the formulation is applied immediately after wounding.

1. A kit for non-surgical, cosmetic use comprising:
one or more growth factors, growth factor mimetics, and/or growth factor receptor agonists formulated for topical application; and
means to perform controlled and multiple wounding of an area of skin, wherein the wounding is targeted to at least the epidermis and/or dermis of the skin to release endogenous thrombin into dermal and/or epidermal tissue.

2. A kit according to claim 1, wherein the means to perform controlled wounding is a mesotherapy device or an array of microneedles.

3. A kit according to claim 1, wherein the means to perform wounding include an ultrasound generator and emitter.

4. A kit according to claim 1, wherein the means to perform controlled wounding include a device to dispense the one or more growth factors, growth factor mimetics, and/or growth factor receptor agonists.

5. A kit according to claim 1, wherein the one or more growth factors is selected from: EGF, VEGF, hb-EGF, PDGF-aa, PDGF-bb, FGF1, FGF2, KGF, HGF, IGF-1, SCF, TGF-alpha, GM-CSF and IFN-gamma.

6. A kit according to claim 5, wherein the one or more growth factors are recombinant and/or synthetic growth factors derived from plant, bacterial, or fungal sources.

7. A kit according to claim 1, wherein the growth factor formulation includes one or more of: superoxide dismutases, superoxide dismutase mimetics, catalases, catalase mimetics and/or antioxidants.

8. A kit according to claim 1, wherein the growth factor formulation includes one or more natural or synthetic nitric oxide donators or mimetics.

9. A kit according to claim 8, wherein the nitric oxide donators or mimetics are selected from minoxidil, tempo, molsidomine and S-nitrosoglutathione.

10. A kit according to claim 1, wherein the growth factor formulation includes a UV protective agent.

11. A kit according to claim 10, wherein the UV protective agent is selected from one or more of propanediol, quaternium-95, polyacrylate-15, titanium dioxide, zinc oxide, ethylhexyl methoxycinnamate, butyl methoxydibenzoylmethane, octyl methoxycinnamate, menthyl anthranilate, homosalate, benzophenone-3 and benzophenone-4.

12. A kit according to claim 1, wherein the formulation includes thrombin.

13. Use of the kit as claimed in claim 1 as part of an autologous growth factor therapy procedure, to reduce hair loss, and/or to maintain or improve hair growth, to reduce the appearance of skin blemishes such as hyper-pigmentation and scarring, to treat or prevent damage to skin cells associated with accumulation of reactive oxygen or nitrogen species such as free radicals and/or after one or more of cosmetic surgery, hair transplant surgery, implantation of dermal filler and/or surgical implants, and/or autologous growth factor therapy to reduce recovery time and/or improve the result.

14. Use of non-surgical controlled and multiple wounding to an area of the skin to sensitise dermal and epidermal tissue to one or more topically applied growth factors, growth factor mimetics, and/or growth factor receptor agonists.
